(19) United States
(12) Patent Application Publication
(10) Pub. No.: US 2015/0223841 A1
(43) Pub. Date: Aug. 13, 2015

(54) Amniotic Membrane Perforator with Integrated Safety Case

(71) Applicant: Kate A. Prendergast, Brooklyn, NY (US)

(72) Inventor: Kate A. Prendergast, Brooklyn, NY (US)

(21) Appl. No.: 14/618,660

(22) Filed: Feb. 10, 2015

Related U.S. Application Data
(63) Continuation-in-part of application No. PCT/US14/46853, filed on Jul. 16, 2014.
(60) Provisional application No. 61/937,896, filed on Feb. 10, 2014.
(60) Provisional application No. 61/937,896, filed on Feb. 10, 2014.

(51) Int. Cl. A61B 17/42

(52) U.S. Cl. CPC A61B 17/4208

(57) ABSTRACT

Amniotomy procedures rupture the amniotic sac as needed in order to accelerate the birthing process and prevent various complications. An amniotic membrane perforator with integrated safety case includes an elongated shaft, a safety hood, and a piercing member to be used in an amniotomy procedure. The piercing member is positioned within the safety hood at one end of the elongated shaft. The amniotic membrane perforator is manually manipulated such that the piercing member and the safety hood interface with amniotic membrane. Using gentle pressure, piercing member is forced through the amniotic membrane, rupturing the amniotic membrane and releasing amniotic fluid. The release of amniotic fluid produces a hormone like effect stimulating contractions to accelerate the birthing process.

The current application is a continuation in part of PCT Patent Application number PCT/US14/46853 filed Jul. 16, 2014 which claims benefit of provisional 61/937,896 filed Feb. 10, 2014. The current application further claims benefit of U.S. Provisional Patent Application 61/937,896 filed Feb. 10, 2014.

### FIELD OF THE INVENTION

The present invention relates generally to an apparatus used by a maternity care provider to rupture the amniotic membrane during childbirth. More specifically, the present invention is an apparatus for an amniotic membrane perforator with integrated safety case.

### BACKGROUND OF THE INVENTION

Millions of childbirths are attended throughout the world each year. Throughout pregnancy, a fetus is surrounded by a liquid within an amnion membrane to cushion and protect the fetus which eventually develops into a child. The amniotic fluid is contained within the two layer amnion/chorion membrane, a part of the placenta. When the mother is going into labor, at some point the amniotic membrane ruptures releasing the fluid, “water breaks”, signaling the child is getting ready to be born. Due to various complications, inducing the birthing process may be needed for the health of the baby as well as the mother. Through a procedure known as an AROM or ARM amniotomy, artificially rupturing of membranes, amniotic fluid is expelled from the amniotic membrane. The fluid causes a release of prostaglandins which provides a hormone-like effect to induce labor.

Therefore, it is an object of the present invention to introduce an apparatus for perforating an amniotic membrane safely for both the child and the mother. The present invention is able to be safely inserted, easily puncture the amniotic membrane, and be easily removed from the patient. The present invention is designed ergonomically for ease of use, while considering the comfort of the mother during the amniotomy procedure.

### BRIEF DESCRIPTION OF THE DRAWINGS

FIG. 1 is a top perspective view of the present invention.

FIG. 2 is a bottom perspective view of the present invention.

FIG. 3 is a front view of the present invention.

FIG. 4 is a side view of the present invention.

FIG. 5 is a top view of the present invention.

FIG. 6 is a side cross-sectional view of the present invention along the line A-A as shown in FIG. 5.

FIG. 7 is a detailed view of the safety hood and the piercing element of the present invention as shown in FIG. 1.

FIG. 8 is a detailed view of the safety hood and the piercing element for an alternate embodiment of the present invention.

FIG. 9 is a detailed view of the safety hood and the piercing element for an alternate embodiment of the present invention as shown in FIG. 2.

FIG. 10 is a detailed view of the safety hood and the piercing element for an alternate embodiment of the present invention as shown in FIG. 3.

FIG. 11 is a detailed view of the safety hood and the piercing element for an alternate embodiment of the present invention as shown in FIG. 4.

FIG. 12 is a detailed view of the safety hood and the piercing element for an alternate embodiment of the present invention as shown in FIG. 6.

FIG. 13 is a perspective view of the present invention being implemented with an amniotic membrane.

FIG. 14 is a perspective view of the present invention as an amniotic membrane is ruptured.

### DETAIL DESCRIPTIONS OF THE INVENTION

All illustrations of the drawings are for the purpose of describing selected versions of the present invention and are not intended to limit the scope of the present invention.

The present invention is an amniotic membrane perforator with integrated safety case to puncture the amniotic membrane in order to induce labor. The present invention is used in amniotomy procedures to artificially rupture the amniotic membrane. Breaking the amniotic membrane releases amniotic fluids which triggers a hormone-like effect to speed up the birthing process by stimulating the strength and frequency of uterine contractions. An amniotomy is the least interventive way to get labor started or to make the labor more progressive and functional. An amniotomy may be necessary to prevent various complications of childbirth, promoting the health and safety of both the mother and the child.

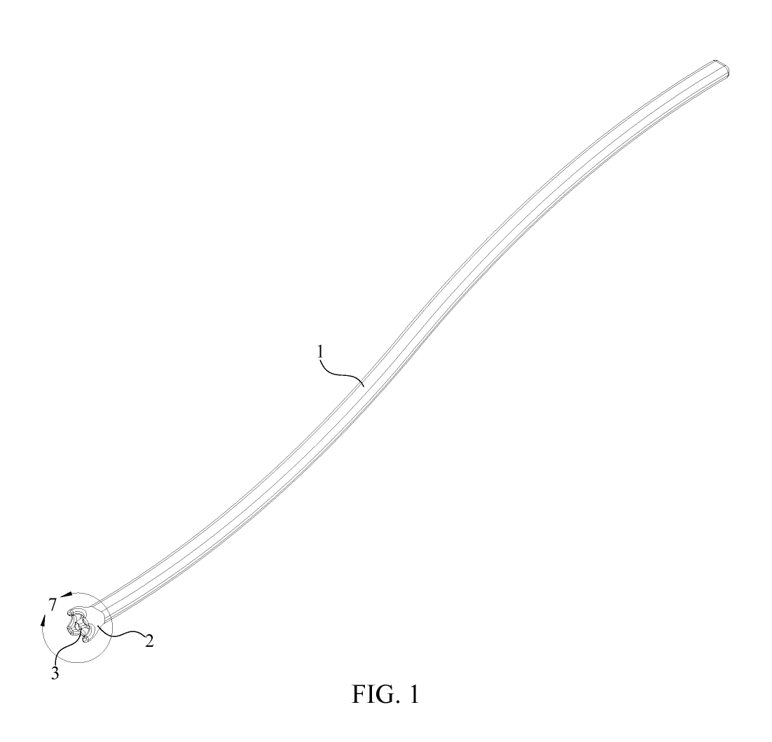

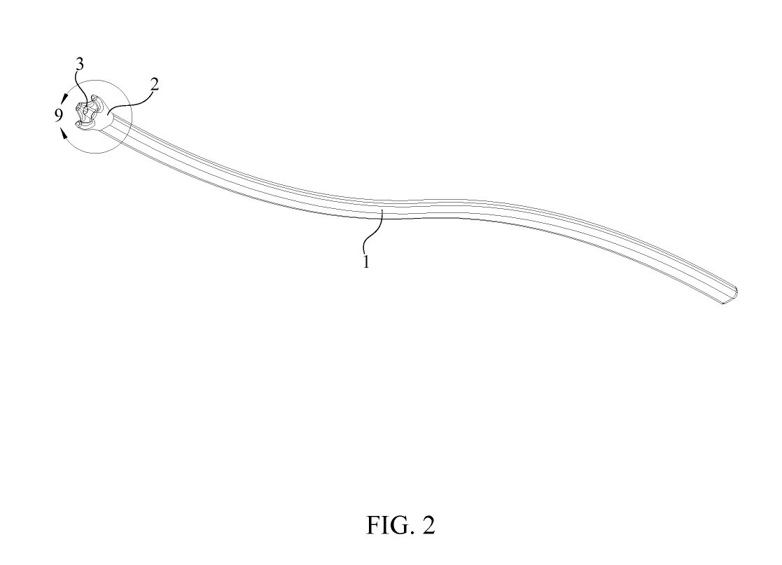

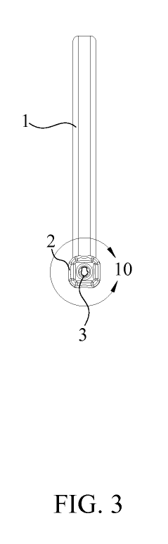

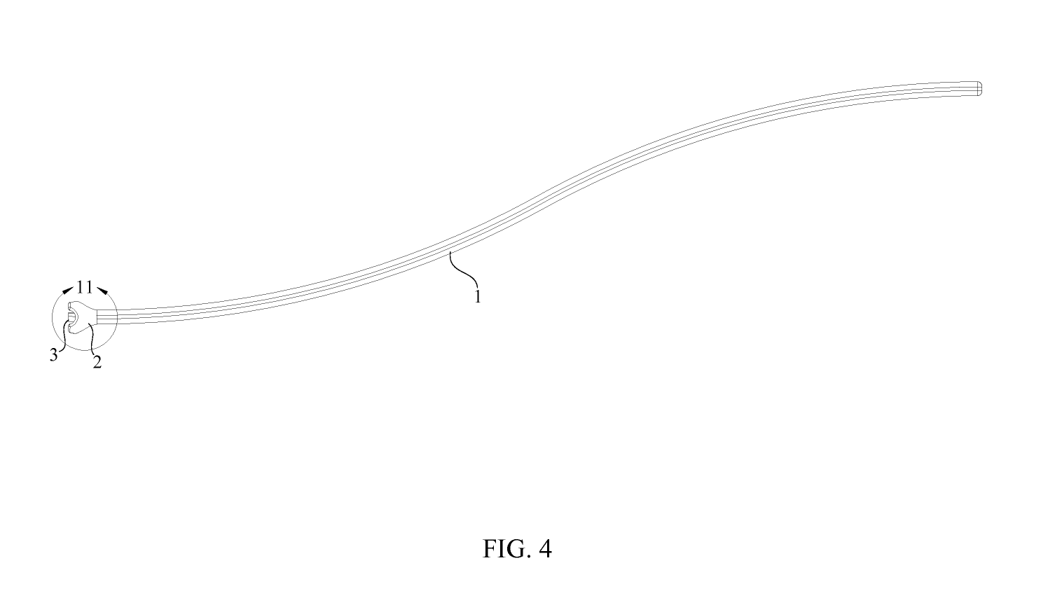

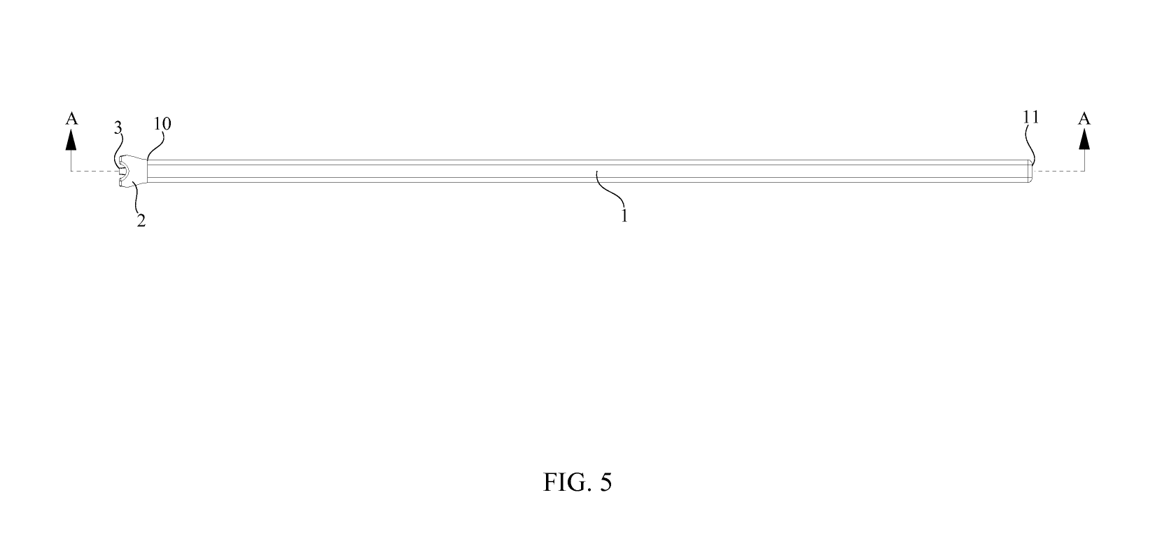

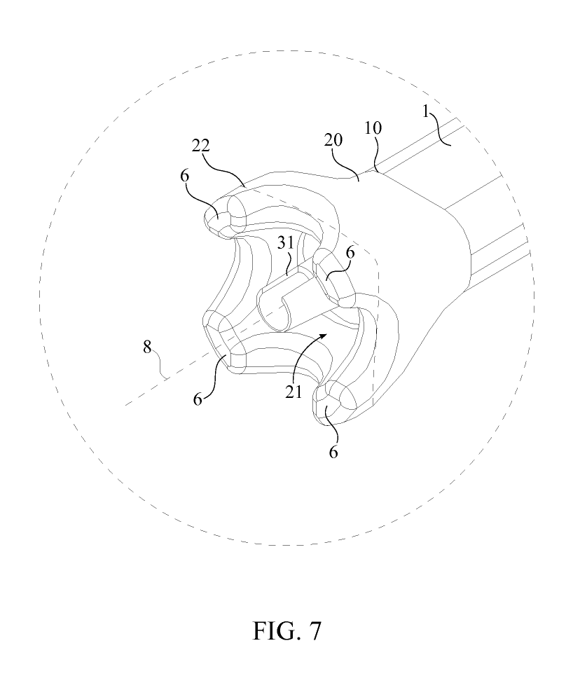

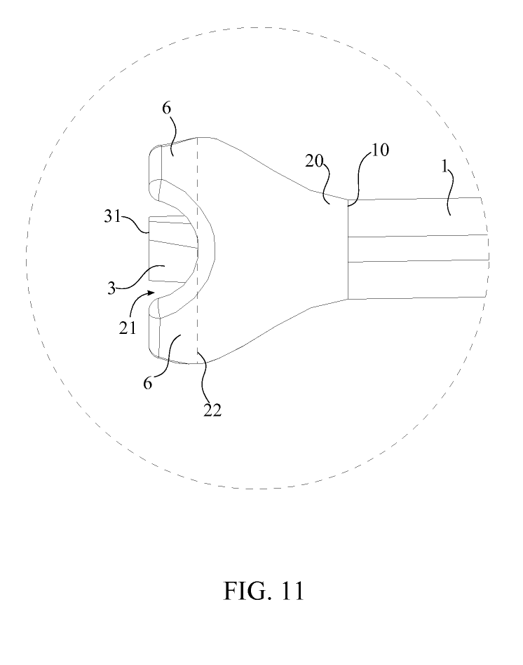

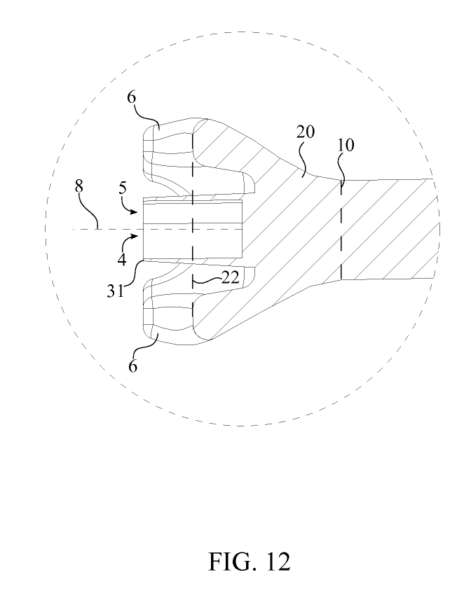

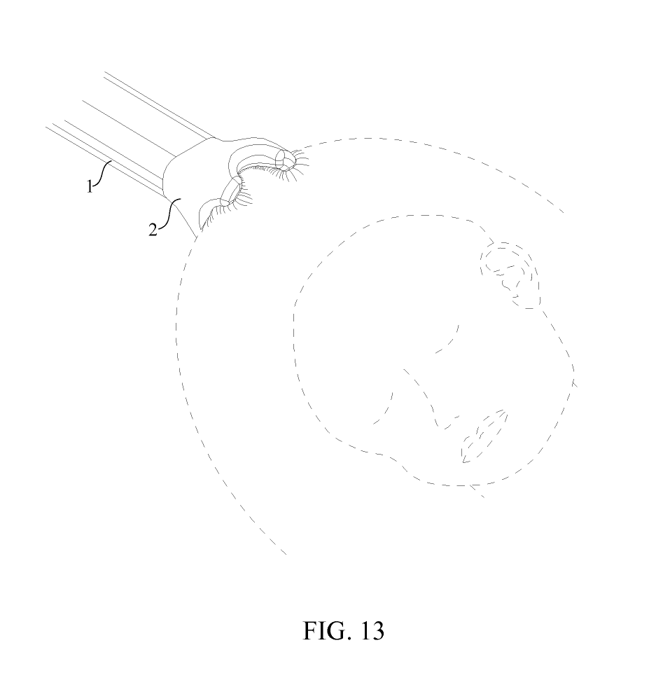

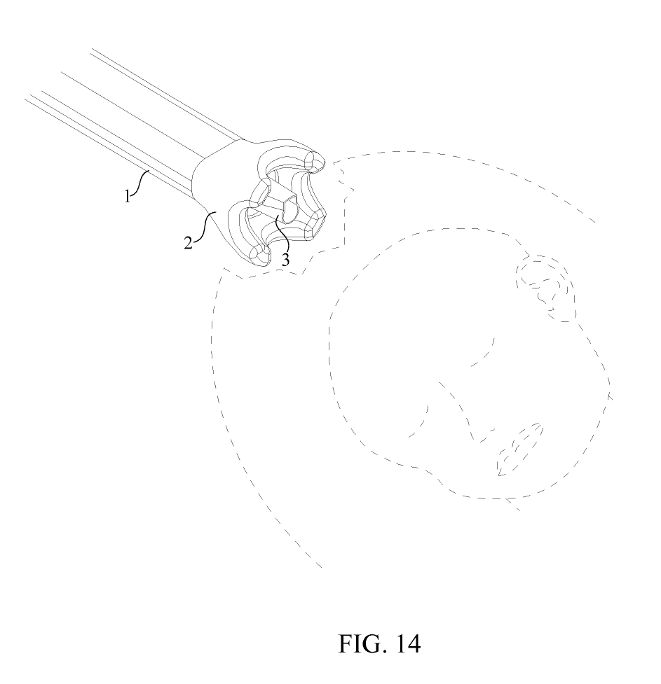

In order to facilitate the amniotomy procedure, the present invention comprises an elongated shaft 1, a safety hood 2, and a piercing member 3, as shown in FIG. 1 to FIG. 6. The elongated shaft 1 allows a user to handle and manipulate the present invention during implementation. The safety hood 2 allows the safe insertion into and removal from the patient. The piercing member 3 ruptures the amniotic membrane when the present invention applies pressure on the amniotic membrane through the intending use. The elongated shaft 1 comprises a first end 10 and a second end 11, as shown in FIG. 5. The first end 10 and the second end 11 are used for reference to position components along the elongated shaft 1. The safety hood 2 comprises a narrow end 20, a wide opening 21, and a membrane interfacing edge 22, as detailed in FIG. 7 to FIG. 9, FIG. 11, and FIG. 12. The narrow end 20 is positioned opposite the membrane interfacing edge 22 through the safety hood 2. The narrow end 20 is adjacently connected to the first end 10. The wide opening 21 is delineated by the membrane interfacing edge 22. The piercing member 3 traverses from the narrow end 20. The piercing member 3 is centrally positioned within the membrane interfacing edge 22. The aforementioned configuration allows the user to apply pressure onto the present invention which transferred through the present invention such that the amniotic membrane deforms around the membrane interfacing edge 22 and onto the piercing member 3. When implemented, the membrane interfacing edge 22 is positioned adjacent to the amniotic membrane through gentle manual manipulation from the user, as shown in FIG. 13. When pressure is applied to the amniotic membrane, the piercing member 3 penetrates the amniotic membrane due to the sharpness of the piercing member 3. As the piercing member 3 penetrates the amniotic membrane, the amniotic fluid is released triggering the hormone like substance to induce labor further and accelerate the process of childbirth, as shown in FIG. 14.

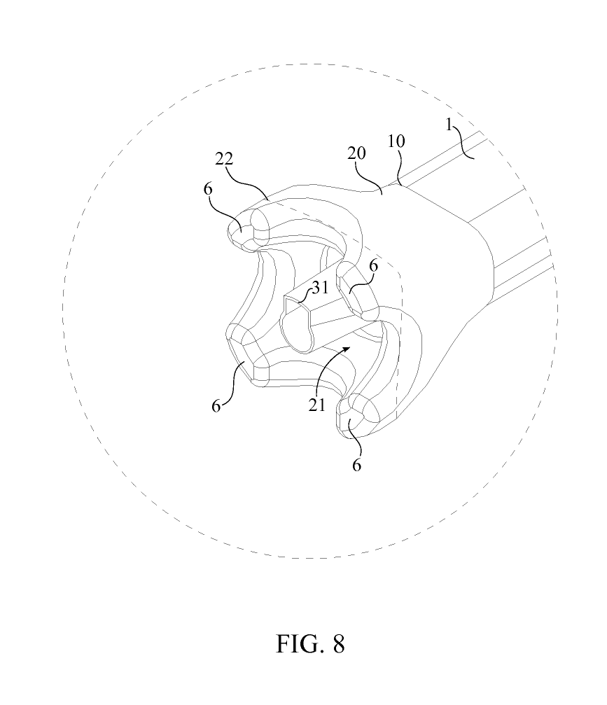

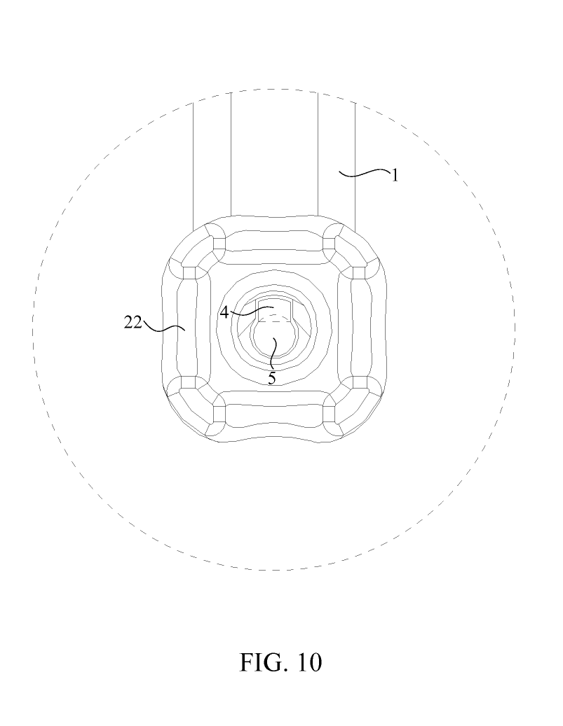

In the preferred embodiment of the present invention, the present invention further comprises a circular cavity 4 and a rectangular cavity 5. A smaller edge of the rectangular cavity 5 intersects the circular cavity 4 as a chord of the circular cavity 4, as shown in FIG. 10. The circular cavity 4 and the rectangular cavity 5 traverse into the piercing member 3 from a distal end 31, in accordance to FIG. 12. In the preferred embodiment, the rectangular cavity 5 traverses through the piercing member perpendicular to a central axis 8, as shown in FIG. 7. In some alternate embodiment, the circular cavity 4 and the rectangular cavity 5 are completely positioned within the piercing member, as shown in FIG. 8 to FIG. 12. This configuration provides an air pocket adjacent to the piercing member 3, which allows the additional space for the amniotic membrane to deform. This deformation causes additional stress on the amniotic membrane causing it to rupture easier. Further in accordance to the preferred embodiment, the piercing member 3 is tapered from the narrow end 20 to the distal end 31. The circular cavity 4, the rectangular cavity 5, and tapering of the piercing member 3 separates the duel layers of the amniotic membrane as present invention is being pressured into the amniotic membrane and prevents the surface tension of the amniotic fluid from re-sealing the puncture.

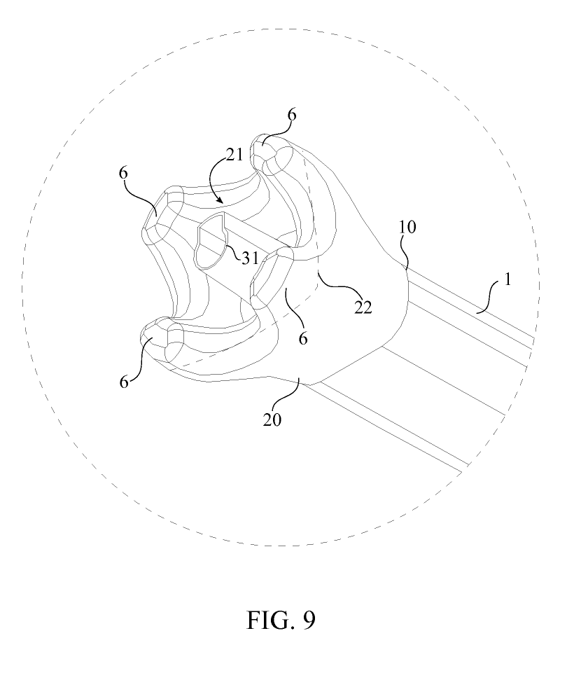

In accordance to the preferred embodiment, the present invention comprises a plurality of extrusions 6, wherein the plurality of extrusions comprises four extrusions, as shown in FIG. 8 and FIG. 9. The plurality of extrusions 6 allows the amniotic membrane to further deform about the safety hood 2. The plurality of extrusions 6 is radially connected and equidistantly distributed about the membrane interfacing edge 22. The plurality of extrusions 6 is oriented parallel to a central axis 8 of the piercing member 3, as shown in FIG. 7 and FIG. 12. In the preferred embodiment of the present invention, the piercing member 3 is flush with the plurality of extrusions 6. The configuration of the preferred embodiment puts pressure at five different points of the amniotic membrane. These pressure points put stress on the surface tension of the amniotic membrane such that the membrane can be easily penetrated by the piercing member 3. The plurality of extrusions 6 and the membrane interfacing edge 22 are rounded and smoothed such that the insertion and manipulation of the present invention does not cause injury to vaginal walls, cervix, or the child. In some embodiments the distal end 31 of the piercing member 3 is flush with the membrane interfacing edge 22, wherein the present invention does not include a plurality of extrusions 6.

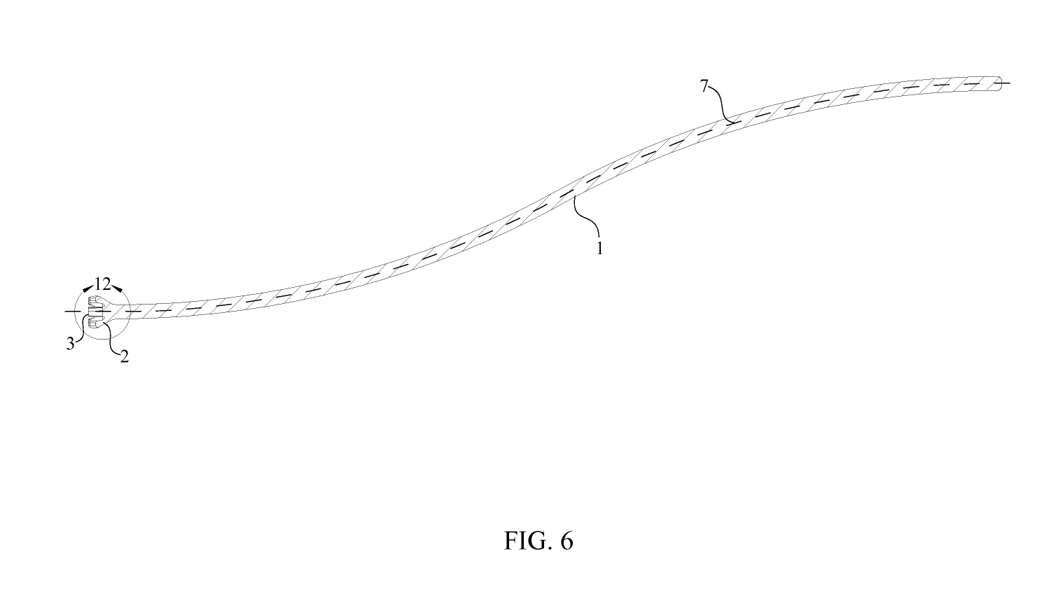

The present invention is also shaped such that the elongated shaft 1 conforms to the female anatomy during the late stages of pregnancy, such that the present invention can be implemented in a safe and relatively comfortable manner. To provide for this shape, the present invention comprises a shape-defining axis 7, in accordance to FIG. 6. The shape defining axis is centrally positioned along the elongated shaft 1 and intersects all cross-sections of the elongated shaft 1. While it is preferred that the shape-defining axis 7 is an S-curve, the shape-defining axis 7 may delineate an elongated shaft 1 of any shape which does not deviate from the spirit of the present invention.

Although the invention has been explained in relation to its preferred embodiment, it is to be understood that many other possible modifications and variations can be made without departing from the spirit and scope of the invention as hereinafter claimed.

What is claimed is:

1. An amniotic membrane perforator with integrated safety case comprises:
an elongated shaft;
a safety hood;
a piercing member;
the elongated shaft comprises a first end and a second end;
the safety hood comprises a narrow end, a wide opening, and a membrane interfacing edge;
the wide opening being delineated by the membrane interfacing edge;
the narrow end being positioned opposite the membrane interfacing edge through the safety hood;
the narrow end being adjacently connected to the first end;
the piercing member traversing out from the narrow end; and
the piercing member being centrally positioned within the membrane interfacing edge.

2. The amniotic membrane perforator with integrated safety case, as claimed in claim 1 comprises:
a circular cavity;
a rectangular cavity;
an edge of the rectangular cavity intersecting the circular cavity as a chord of the circular cavity; and
the rectangular cavity and the circular cavity traversing into the piercing member from a distal edge of the piercing member.

3. The amniotic membrane perforator with integrated safety case, as claimed in claim 1 comprises:
the piercing member tapering from the narrow end to a distal end of the piercing member.

4. The amniotic membrane perforator with integrated safety case, as claimed in claim 1 comprises:
a plurality of extrusions;
the plurality of extrusions being radially connected about the membrane interfacing edge;
the plurality of extrusions being equidistantly distributed around the membrane interfacing edge; and
each of the plurality of extrusions being oriented parallel to a central axis of the piercing member.

5. The amniotic membrane perforator with integrated safety case, as claimed in claim 5 comprises:
the piercing member being flush with the plurality of extrusions.

6. The amniotic membrane perforator with integrated safety case, as claimed in claim 1 comprises:
a shape-defining axis;
the shape-defining axis being centrally positioned along the elongated shaft; and
the shape-defining axis being an S-curvature.

7. An amniotic membrane perforator with integrated safety case comprises:
an elongated shaft;
a safety hood;
a piercing member;
a plurality of extrusions;
the elongated shaft comprises a first end and a second end;
the safety hood comprises a narrow end, a wide opening, and a membrane interfacing edge;
the wide opening being delineated by the membrane interfacing edge;
the narrow end being positioned opposite the membrane interfacing edge through the safety hood;
the narrow end being adjacently connected to the first end;
the piercing member traversing out from the narrow end;
the piercing member being centrally positioned within the membrane interfacing edge;
the plurality of extrusions being radially connected about the membrane interfacing edge;
the plurality of extrusions being equidistantly distributed around the membrane interfacing edge; and
each of the plurality of extrusions being oriented parallel to a central axis of the piercing member.

8. The amniotic membrane perforator with integrated safety case, as claimed in claim 7 comprises:
a circular cavity;
a rectangular cavity;
an edge of the rectangular cavity intersecting the circular cavity as a chord of the circular cavity; and
the rectangular cavity and the circular cavity traversing into the piercing member from a distal edge of the piercing member.

9. The amniotic membrane perforator with integrated safety case, as claimed in claim 7 comprises:
the piercing member tapering from the narrow end to a distal end of the piercing member.

10. The amniotic membrane perforator with integrated safety case, as claimed in claim 7 comprises:
the piercing member being flush with the plurality of extrusions.

11. The amniotic membrane perforator with integrated safety case, as claimed in claim 7 comprises:
a shape-defining axis;
the shape-defining axis being centrally positioned along the elongated shaft; and
the shape-defining axis being an S-curvature.

12. An amniotic membrane perforator with integrated safety case comprises:
an elongated shaft;
a safety hood;
a piercing member;
a circular cavity;
a rectangular cavity;
the elongated shaft comprises a first end and a second end;
the safety hood comprises a narrow end, a wide opening, and a membrane interfacing edge;
the wide opening being delineated by the membrane interfacing edge;
the narrow end being positioned opposite the membrane interfacing edge through the safety hood;
the narrow end being adjacently connected to the first end;
the piercing member traversing out from the narrow end;
the piercing member being centrally positioned within the membrane interfacing edge;
an edge of the rectangular cavity intersecting the circular cavity as a chord of the circular cavity; and
the rectangular cavity and the circular cavity traversing into the piercing member from a distal edge of the piercing member.

13. The amniotic membrane perforator with integrated safety case, as claimed in claim 12 comprises:
the piercing member tapering from the narrow end to a distal end of the piercing member.

14. The amniotic membrane perforator with integrated safety case, as claimed in claim 12 comprises:
a plurality of extrusions;
the plurality of extrusions being radially connected about the membrane interfacing edge;
the plurality of extrusions being equidistantly distributed around the membrane interfacing edge; and
each of the plurality of extrusions being oriented parallel to a central axis of the piercing member.

15. The amniotic membrane perforator with integrated safety case, as claimed in claim 14 comprises:
the piercing member being flush with the plurality of extrusions.

16. The amniotic membrane perforator with integrated safety case, as claimed in claim 12 comprises:
a shape-defining axis;
the shape-defining axis being centrally positioned along the elongated shaft; and
the shape-defining axis being an S-curvature.
